(19) United States
(12) Patent Application Publication
(10) Pub. No.: US 2014/0342945 A1
(43) Pub. Date: Nov. 20, 2014

(54) FORENSIC METHOD

(71) Applicant: Eberhard-Karls-Universitaet Tuebingen Universitaetsklinikum, Tuebingen (DE)

(72) Inventors: Sascha DAMMEIER, Tuebingen (DE); Heinz-Dieter WEHNER, Tuebingen (DE)

(21) Appl. No.: 14/341,321

(22) Filed: Jul. 25, 2014

Related U.S. Application Data
(63) Continuation of application No. PCT/EP2013/051694, filed on Jan. 29, 2013.

(30) Foreign Application Priority Data
Jan. 31, 2012 (DE) 10 2012 100 781.0

(51) Int. Cl. G01N 33/68

(52) U.S. Cl. CPC G01N 33/6848; G01N 33/6887; G01N 33/6884
USPC 506/12

(57) ABSTRACT

The present invention relates to a forensic method for identifying an organ penetrated by a corpus delicti.

### CROSS-REFERENCE TO RELATED APPLICATIONS

This application is a continuation of copending international patent application PCT/EP2013/051694 filed on 29 Jan. 2013 and designating the U.S., which has been published in German, and claims priority from German patent application DE 10 2012 100 781.0 filed on 31 Jan. 2012. The entire contents of these prior applications are incorporated herein by reference.

### FIELD

The present invention relates to a forensic method for identifying an organ penetrated by a corpus delicti.

### BACKGROUND

In the forensic examination of crime victims it is of central importance to detect with high reliability which injuries were caused by an object, such as a cut or thrust weapon or a gun, in particular if this injury led to the death of the victim. This applies especially for the reconstruction of the circumstances of the offence in a case where the crime victim has been injured or killed by several objects, respectively. In this case it is required to provide evidence demonstrating which object has caused the lethal injury.

By the means of modern molecular biological methods it is possible to allocate genetical material which remained on an object to the injured or killed person. However, it is not or only hardly possible to allocate this material accurately to a specific organ.

The forensic methods described in the art are generally immunocytochemical examinations. Wehner et al. (2008), Immunocytochemical examinations of biological traces on expanding bullets (QD-PEP), Forensic Science International 182, pages 66-70, describe that tissue from a swine adhering to a bullet can be successfully allocated to the heart and the liver. The detection was realized by the use of an antibody specific for mitochondria of hepatocytes, having the designation HepPar 1, and an anti-body specific for heart troponin 1 having the designation DAKO. However, this method has the fundamental disadvantage that it depends from the availability of highly selective antibodies. In addition, for every organ-specific analysis one has to re-access to the biological material. This requires a large experimental investment and causes difficulties in cases where only small amounts of the biological materials are available. Furthermore, it turned out that the known method is prone to error and very often a reliable allocation of the tissue adhering to the bullet to a penetrated organ is not possible with sufficient certainty. This complicates criminalistic investigative work and may finally have the result that it might not be possible to identify the perpetrator.

### SUMMARY

Against this background an object underlying the invention is to provide a forensic method for identifying an organ penetrated by a corpus delicti, by means of which the disadvantages of the prior art can at least in parts be avoided. Especially, such a forensic method should be provided which is characterized by a high specificity and analytical certainty.

This object is met by a forensic method comprising the following steps:
1) providing of biological material which was in contact with the corpus delicti,
2) establishing a total protein profile of the biological material,
3) analyzing of the total protein profile for an organ-specific protein signature,
4) identifying of the organ penetrated by the corpus delicti in case of the presence of an organ-specific protein profile.

According to the invention a corpus delicti refers to a subject matter or an object of crime, respectively. Examples are cut and thrust weapons or guns or gun bullets etc.

According to the invention “total protein profile” refers to a pattern that is representative for the entire proteins of the biological material.

According to the invention, “organ-specific protein signature” refers to such a pattern which represents at least one or more proteins which can be allocated to a specific organ. According to the invention, such proteins are referred to as “organ-specific proteins”.

Organ-specific proteins are well-known to the skilled person and are for example described in WO 2008/021290. This document further describes methods for determining such organ-specific proteins. The content of this document is incorporated herein by reference.

It is to be understood that by means of the method according to the invention not only one organ but several organs can be identified which were penetrated by a corpus delicti. The singular form is only used for reasons of a better readability.

Hereby the problem underlying the invention is completely solved.

The method according to the invention has the advantage that it allows a search for a plurality of different organ-specific protein signatures with high accuracy without having the need for the availability of antibodies. The examination starts from a total protein profile of the biological material. It can be systematically analyzed for a plurality of different organs without a re-access to the biological material. For this reason the method according to the invention requires only little amounts of biological material. The determination of an organ-specific protein signature results in a higher specificity and a greater analytical certainty than by using immunocytochemical methods.

In another embodiment of the method according to the invention the organ-specific protein signature is a brain-specific, heart-specific, liver-specific, lung-specific, kidney-specific, and/or skeletal muscle-specific protein signature. This measure has the advantage that by means of the mentioned protein signatures an allocation of the biological material to such organs becomes possible which are, from a forensic point of view, the most important organs.

In another embodiment of the method according to the invention the organ-specific protein signature comprises at least 2, preferably 3, further preferably 4, further preferably 5, further preferably 6, further preferably 7, further preferably 8, further preferably 9, further preferably 10 or more organ-specific proteins.

This measure has the advantage that with an increasing number of organ-specific proteins an increasing specificity of the protein signature can be reached. By doing so, the method according to the invention achieves an even higher analytical certainty.

In another embodiment of the method according to the invention the
a. brain-specific protein signature comprises brain-specific proteins which are selected from the group consisting of: myelin basic protein (MBP), myelin oligodendrocyte glycoprotein (MOG), myelin-associated oligodendrocytic basic glycoprotein (MOBP) and the AT1 A3-Na/K-ATPase-alpha-3-subunit, and/or
b. heart-specific protein signature comprises heart-specific proteins which are selected from the group consisting of: myosin binding protein C (MYBPC3), troponin C (TNNC1), ventricular/myocardial muscle isoform of the myosin regulatory light chain 2 (MYL2), troponin I (TNNI3), AT1 A1-Na/K-ATPase-alpha-1-subunit (AT1 A1), creatin kinase S type (KCRS), citrate synthase (CISY), and isocitrate dehydrogenase (IDHP), and/or the
c. liver-specific protein signature comprises liver-specific proteins which are selected from the group consisting of: aspartate aminotransferase (AATM), 4-aminobutyrate aminotransferase (GABT), serine-pyruvate aminotransferase (SPYA), alcohol dehydrogenase (ADH) , carbamoyl-phosphate synthase (CPSM), glycogen phosphorylase (PYGL), copper transport protein (ATOX1), phospholysine phosphohistidine inorganic pyrophosphate phosphatase (LHPP), proteasome subunit alpha type-2 (PSMA2), proteasome subunit alpha type-6 (PSMA6), glycine N-acyltransferase (GLYAT), glycine amidinotransferase (GATM) , arginase 1 (ARGI1), prostaglandine F synthase (PGFS), prostaglandine reductase (PTGR1) and argininosuccinate lyase (ARLY), and/or the
d. lung-specific protein signature comprises lung-specific proteins which are selected from the group consisting of: pulmonary surfactant associated protein A1 (SFPA1), pulmonary surfactant associated protein A1 (SFPA2), pulmonary surfactant associated protein B (PSPB), cathelicidine antimicrobial peptide (CAMP), annexin A1/A3/A2 (ANXA), transgelin 2 (TAGLN), histones H1, H2b, H4 (H2, H4), 14-3-3 beta/alpha/delta (1433B), and/or the
e. skeletal muscle-specific protein signature comprises skeletal muscle specific proteins which are selected from the group consisting of: actin/alpha skeletal muscle (ACTA), troponin C/skeletal muscle (TNNC), troponin T/fast skeletal muscle (TNNT), myosin light-chain ⅓ skeletal muscle isoform (MLC), myosin regulatory light-chain 2 skeletal muscle isoform (MLRS), myosin 1 (MYH1), myosin 2 (MYH2), aldolase C fructose-bisphosphate (ALDOC), aldolase A fructose-bisphosphate (ALDO), myoglobin (MYG), and/or the
f. kidney-specific protein signature comprises kidney-specific proteins selected from the group consisting of: acid ceramidase (ASAH1), glycine amidinotransferase (GATM), chloride intracellular channel protein (CLIC4), transketolase (TKT), voltage-dependent anion-selective channel (VDAC), aquaporine 1 (AQP1).

This measure has the advantage that—depending on the organ—such proteins are analyzed, which allow a highly specific allocation of the biological material.

In another embodiment of the method according to the invention steps 2 and 3 are performed by means of a mass spectrometric analysis.

This measure has the advantage that a method comes into operation which is established in the protein analysis and which allows a determination of a plurality of organ-specific protein signatures with high specificity and in a widely automized manner.

In another embodiment of the method according to the invention the total protein profile of the biological material is established in step 2 as follows:
mass spectrometric analysis of the total protein of the biological material to obtain mass spectra, and
comparison of the obtained mass spectra with a protein database to obtain the total protein profile in form of a pattern which identifies individual proteins.

By this measure the comprehensive data pool of established bioinformatic databases is used to determine on the basis of the generated mass spectra the proteins which are contained in the biological material. The establishment of a pattern which identifies individual proteins is realized automatically by using adequate software such as e.g. Mascot (Matrix Sciences). By the obtainment of a pattern which identifies individual proteins, the starting point for the subsequent determination of the organ-specific protein signature is created. Examples for suitable protein databases are UniProt, Swiss-Prot, TrEMBL, and Protein Information Resource (PIR).

In another embodiment of the method according to the invention the pattern which identifies individual proteins is analyzed for the presence of the organ-specific protein signature.

By this measure the determination of the organ-specific protein signature is realized in an advantageous manner. The identification is still realized automatically, e.g. by using the Mascot software.

Against this background the present invention also relates to the use of a organ-specific protein signature of a biological material that was in contact with a corpus delicti for the identification of an organ penetrated by the corpus delicti.

The features, advantages and preferred further developments of the method according to the invention apply to the use according to the invention correspondingly.

It is to be understood that the features mentioned before and those to be mentioned in the following cannot only be used in each of the indicated combination but also in other combinations or in isolated manner without departing from the scope of the present invention.

The invention is now explained in more detail by means of embodiments resulting in further features and advantages of the invention. The embodiments are for the purpose of illustrating the invention and do not limit the scope of protection.

In the embodiments reference is made to the enclosed figures.

### BRIEF DESCRIPTION OF THE DRAWINGS

FIG. 1 (A-D) shows the organ- and tissue-specific mRNA expression profiles of markers from Affymetrix experiments;

FIG. 2 shows the result of a preliminary experiment where a bullet sample which before has penetrated human cardiac tissue is analyzed by means of the method according to the invention;

FIG. 3 shows the result of an experiment where blinded bullet samples which before have penetrated different tissues were analyzed by means of the method according to the invention.

### EXAMPLES

### Example 1

### Organ-Specific Proteins

In the following tables the most important organ-specific proteins are listed having specificities or primary specificities for the organs of heart, liver, lung, skeletal muscle, and kidney. In addition references are provided where the respective organ specificity is described. Furthermore, for each organ-specific protein the short-term as used in the databases (“accession number) as well as a secondary specificity, if any, as described in the literature is provided.

TABLE 1
Proteins with the organ specificity ″brain″

| | | | Secondary | |
|---|---|---|---|---|
| Protein | Name | Accession No. | Specificity* | Reference |
| MBP | myelin basic | MBP_HUMAN | CNS | http://www.uniprot.org/uniprot/ |
| | protein | | | P02686 |
| MOG | myelin | MOG_HUMAN | CNS | http://www.uniprot.org/uniprot/ |
| | oligodendrocyte | | | Q16653 |
| | glycoprotein | | | |
| MOBP | myelin- | MOBP_HUMAN | CNS | http://www.uniprot.org/uniprot/ |
| | associated | | | Q13875 |
| | oligodendrocytic | | | |
| | basic glycoprotein | | | |
| AT1A3 | AT1A3-Na/K- | AT1A3_HUMAN | CNS | De Carvalho Aguiar P. et al. |
| | ATPase-alpha- | | | (2004), Neuron 43: 169-175 |
| | 3-subunit | | | |

*according to literature

TABLE 2
Proteins with the organ specificity ″heart″

| | | | Secondary | |
|---|---|---|---|---|
| Protein | Name | Accession No. | Specificity* | Reference |
| MYBPC3 | myosin binding | MYPC3_HUMAN | | http://www.uniprot.org/uniprot/ |
| | protein C | | | Q14896 |
| TNNC1 | troponin C | TNNC1_HUMAN | muscle | Sogah VM et al. (2010), Dev Dyn |
| | | | | 239(11): 3115-23. |
| MYL2 | ventricular/myocardial | MLRV_HUMAN | | http://www.uniprot.org/uniprot/ |
| | muscle isoform of the | | | P10916 |
| | myosin regulatory | | | |
| | light chain 2 | | | |
| TNNI3 | troponin I | TNNI3_HUMAN | | http://www.uniprot.org/uniprot/ |
| | | | | P19429 |
| AT1A1 | AT1A1-Na/K- | AT1A1_HUMAN | kidnney, | |
| | ATPase-alpha-1- | | CNS | |
| | subunit | | | |
| KCRS | creatin kinase S type | KCRS_HUMAN | muscle | http://www.uniprot.org/uniprot/ |
| | | | | P17540 |
| CISY | citrate synthase | CISY_HUMAN | muscle | Siu PM. et al. (2003), J Appl |
| | | | (kidney) | Physiol. 94(2): 555-60. |
| IDHP | isocitrate | IDHP_HUMAN | kidney | |
| | dehydrogenase | | | |

*according to literature

TABLE 3
Proteins with the organ specificity ″liver″

| | | | Secondary | |
|---|---|---|---|---|
| Protein | Name | Accession No. | Specificity* | Reference |
| AATM | aspartate | AATM_HUMAN | heart | http://www.ebi.ac.uk/gxa/ |
| | aminotransferase | | | gene?gid=P00505 |
| GABT | 4-aminobutyrate | GABT_HUMAN | pancreas | http://www.uniprot.org/ |
| | aminotransferase | | | uniprot/P80404 |
| SPYA | serine-pyruvate | SPYA_HUMAN | | http://www.uniprot.org/ |
| | aminotransferase | | | uniprot/P21549 |
| ADH | alcohol dehydrogenase | ADH4_HUMAN | | http://www.ebi.ac.uk/gxa/ |
| | | | | gene?gid=P08319 |
| | alcohol dehydrogenase | ADH1A_HUMAN | | |
| CPSM | carbamoyl-phosphate | CPSM_HUMAN | small | http://www.uniprot.org/ |
| | synthase | | intestine | uniprot/P31327 |
| PYGL | glycogen phosphorylase | PYGL_HUMAN | | http://www.uniprot.org/ |
| | | | | uniprot/P06737 |
| ATOX1 | copper transport | ATOX1_HUMAN | | |
| | protein | | | |
| LHPP | phospholysine | LHPP_HUMAN | kidney | http://www.uniprot.org/ |
| | phosphohistidine | | | uniprot/Q9H008 |
| | inorganic | | | |
| | pyrophosphate | | | |
| | phosphatase | | | |
| PSMA2 | proteasome subunit | PSA2_HUMAN | | |
| | alpha type-2 | | | |
| PSMA6 | proteasome subunit | PSA6_HUMAN | | |
| | alpha type-6 | | | |
| GLYAT | glycine N- | GLYAT_HUMAN | kidney | http://www.ebi.ac.uk/gxa/ |
| | acyltransferase | | | gene?gid=P50440 |
| GATM | glycine | GATM_HUMAN | kidney | |
| | amidinotransferase | | | |
| ARGI1 | arginase 1 | ARGI1_HUMAN | | http://www.ebi.ac.uk/gxa/ |
| | | | | gene?gid=P05089 |
| PGFS | prostaglandine F | PGFS_HUMAN | | |
| | synthase | | | |
| PTGR1 | prostaglandine | PTGR1_HUMAN | kidney | http://www.uniprot.org/ |
| | reductase | | | uniprot/Q14914 |
| ARLY | argininosuccinate lyase | ARLY_HUMAN | kidney, | http://www.ebi.ac.uk/gxa/ |
| | | | intestine | gene?gid=P04424 |

*according to literature

TABLE 4
Proteins with the organ specificity ″lung″

| | | | Secondary | |
|---|---|---|---|---|
| Protein | Name | Accession No. | Specificity* | Reference |
| SFPA1 | pulmonary | SFTA1_HUMAN | | http://www.uniprot.org/uniprot/ |
| | surfactant | | | Q8IWL2 |
| | associated | | | |
| | protein A1 | | | |
| SFPA2 | pulmonary | SFPA2_HUMAN | | http://www.uniprot.org/uniprot/ |
| | surfactant | | | Q8IWL1 |
| | associated | | | |
| | protein A1 | | | |
| PSPB | pulmonary | PSPB_HUMAN | | http://www.uniprot.org/uniprot/ |
| | surfactant | | | P07988 |
| | associated | | | |
| | protein B | | | |
| CAMP | cathelicidine | CAMP_HUMAN | bone | |
| | antimicrobial | | marrow | |
| | peptide | | | |
| ANXA | annexin | ANXA2_HUMAN | stomach | |
| | A1/A3/A2 | | | |
| TAGLN | transgelin 2 | TAGL2_HUMAN | liver | http://www.ebi.ac.uk/gxa/ |
| | | | | gene?gid=P37802 |
| H2, H4 | histones H1, | H2B1C_HUMAN | liver, heart | |
| | H2b, H4 | | | |
| 1433B | 14-3-3 | 1433B_HUMAN | | |
| | beta/alpha/delta | | | |

*according to literature

TABLE 5
Proteins with the organ specificity ″skeletal muscle″

| | | | Secondary | |
|---|---|---|---|---|
| Protein | Name | Accession No. | Specificity* | Reference |
| ACTA | actin/alpha | ACTS_HUMAN | | |
| | skeletal muscle | | | |
| TNNC | troponin | TNNC2_HUMAN | | http://www.uniprot.org/ |
| | C/skeletal | | | uniprot/P02585 |
| | muscle | | | |
| TNNT | troponin T/fast | TNNT3_HUMAN | | http://www.uniprot.org/ |
| | skeletal muscle | | | uniprot/P45378 |
| MLC | myosin light- | MYL1_HUMAN | | http://www.uniprot.org/ |
| | chain 1/3 | | | uniprot/P05976 |
| | skeletal muscle | | | |
| | isoform | | | |
| MLRS | myosin | MLRS_HUMAN | | http://www.uniprot.org/ |
| | regulatory light- | | | uniprot/Q96A32 |
| | chain 2 skeletal | | | |
| | muscle isoform | | | |
| MYH1 | myosin 1 | MYH1_HUMAN | | http://www.uniprot.org/ |
| | | | | uniprot/P12882 |
| MYH2 | myosin 2 | | | |
| ALDOC | aldolase C | ALDOC_HUMAN | brain | |
| | fructose- | | | |
| | bisphosphate | | | |
| ALDO | aldolase A | ALDOA_HUMAN | | http://www.ncbi.nlm.nih. |
| | fructose- | | | gov/gene/226 |
| | bisphosphate | | | |
| MYG | myoglobin | MYG_HUMAN | heart, lung | |

*according to literature

TABLE 6
Proteins with the organ specificity ″kidney″

| | | | Secondary | |
|---|---|---|---|---|
| Protein | Name | Accession No. | Specificity* | Reference |
| ASAH1 | acid ceramidase | ASAH1_HUMAN | heart | |
| GATM | glycine | GATM_HUMAN | liver | http://www.uniprot.org/ |
| | amidinotransferase | | | uniprot/P50440 |
| CLIC4 | chloride intracellular | CLIC4_HUMAN | heart, | |
| | channel protein? 4 | | muscle | |
| TKT | transketolase | TKT_HUMAN | liver, brain | |
| VDAC | voltage-dependent | VDAC1_HUMAN | brain | |
| | anion-selective channel | | | |
| AQP1 | aquaporine 1 | AQP1_HUMAN | | http://en.wikipedia.org/ |
| | | | | wiki/Aquaporin 1 |

*according to literature

### Example 2

### Preliminary Experiment

In a preliminary experiment it was intended to clarify whether on a bullet, which has penetrated a human heart before, organ-specific proteins of the heart can be detected.

Sample Isolation/Preparation

In the course of an autopsy a bullet of a gun was mechanically pushed through a human heart. The bullet was recovered in a sample bag and stored at -20° C. until further processing. For the analysis the metal ball was removed and subjected to a suitable plastic vial for a proteolytic cleavage by means of the protease trypsin. For this purpose, in a solution first a reduction with dithiothreitol and subsequently an alkylation with iodoacetamide is performed before the protease is added and incubated over night at 37° C. Because of adhering tissue residues, the interior of the sample bag was also incubated with the reagents for the tryptic cleavage and treated like the metal ball. The reaction was stopped by trifluoroacetic acid (final concentration: 5%) and the resulting peptide solution was centrifuged. After the concentration via stage tips (Proxeon) a part of the supernatant was separated on an UltiMate 3000 Nano HPLC installation (Dionex) via a Acclaim PepMap RSLC 75μ×25 cm C18 2 μm 100 A.

Establishing a Total Protein Profiel by Mass Spectrometry

The concentrated and chromatographically separated supernatants of the peptide solutions were measured online in a LTQ Orbitrap mass spectrometer (Thermo Fischer Scientific). In each case the 10 most intensive masses of a filling cycle were automatically selected for a fragmentation. The fragmentation was done by means of the CID method (collision induced decay). The masses appearing in the fragment spectra were compared with the theoretic protein and peptide mass parameters from the SwissProt databse by using the Mascot search algorithm (Matrix Science) and the results were visualized by the aid of the Scaffold software (Proteome Software Inc.). The result is a total protein profile in the form of so-called hit or result lists where the protein identifications were weighted with respect to their reliability or non-accidental appearance.

Analysis of the Total Protein Profile for a Heart-Specific Protein Signature

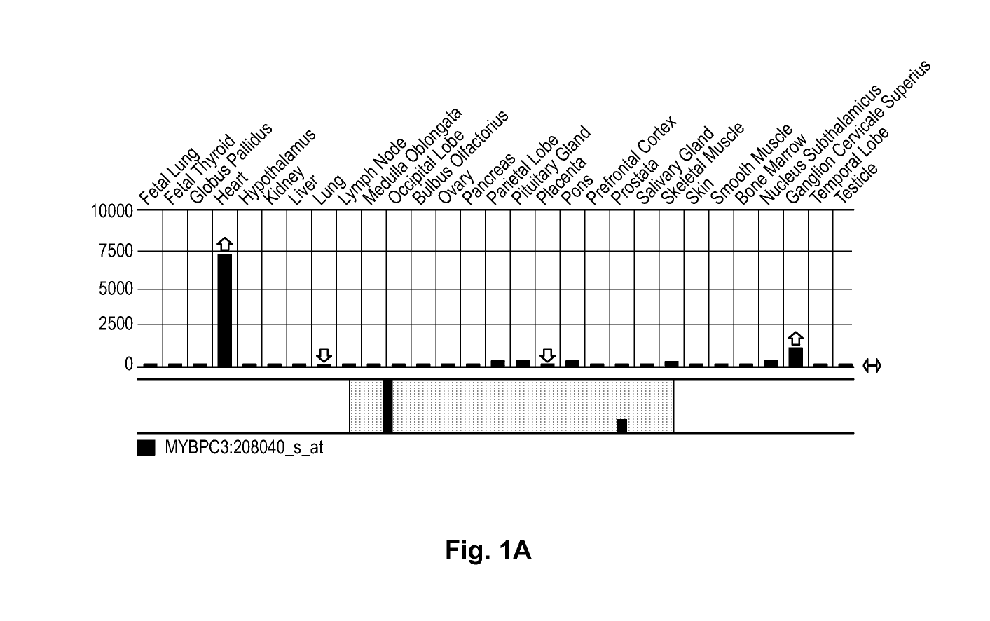

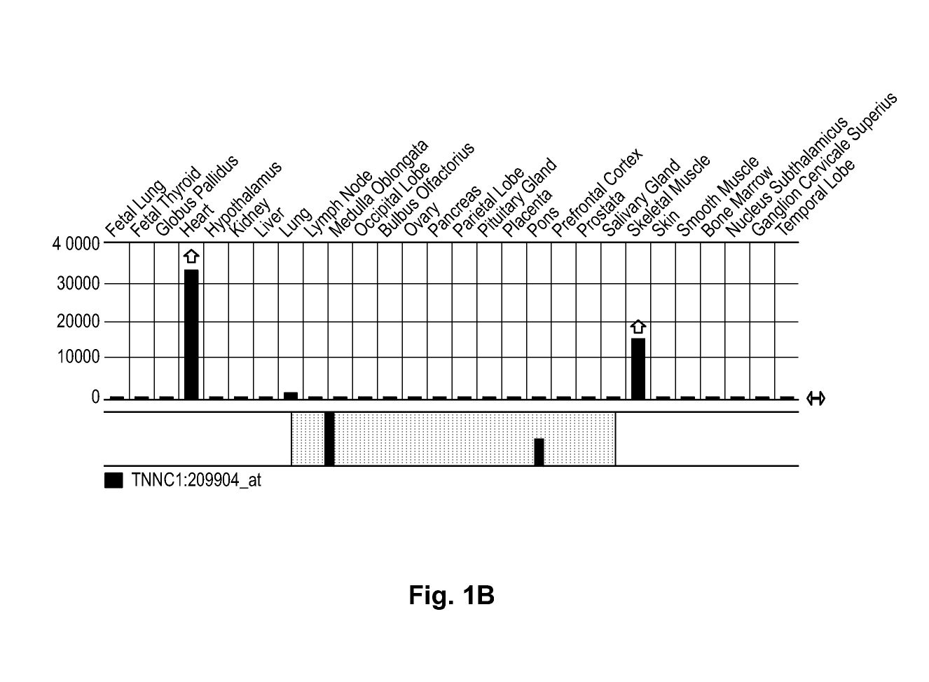

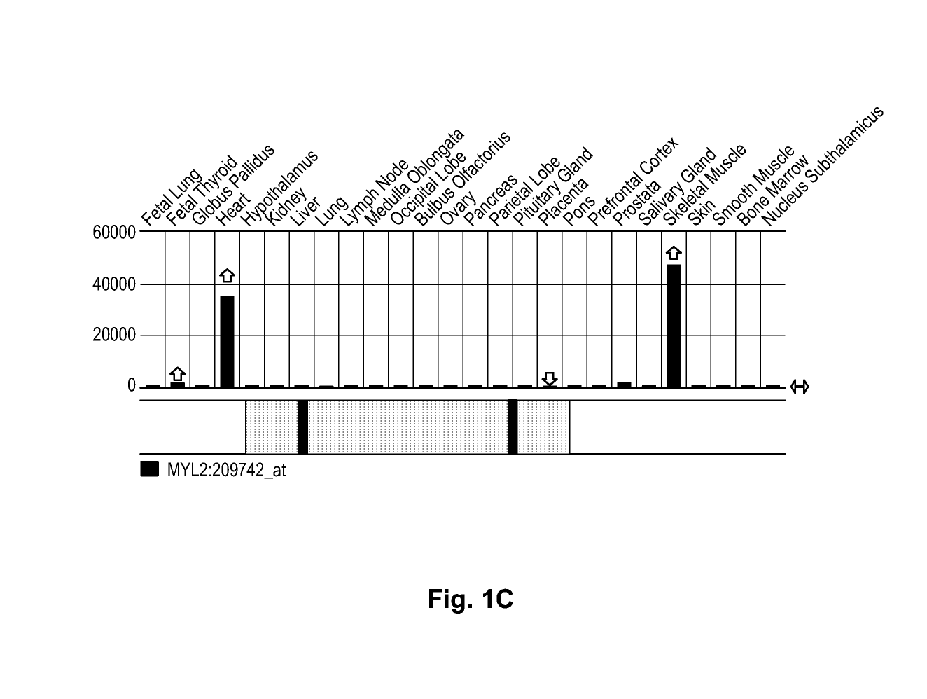

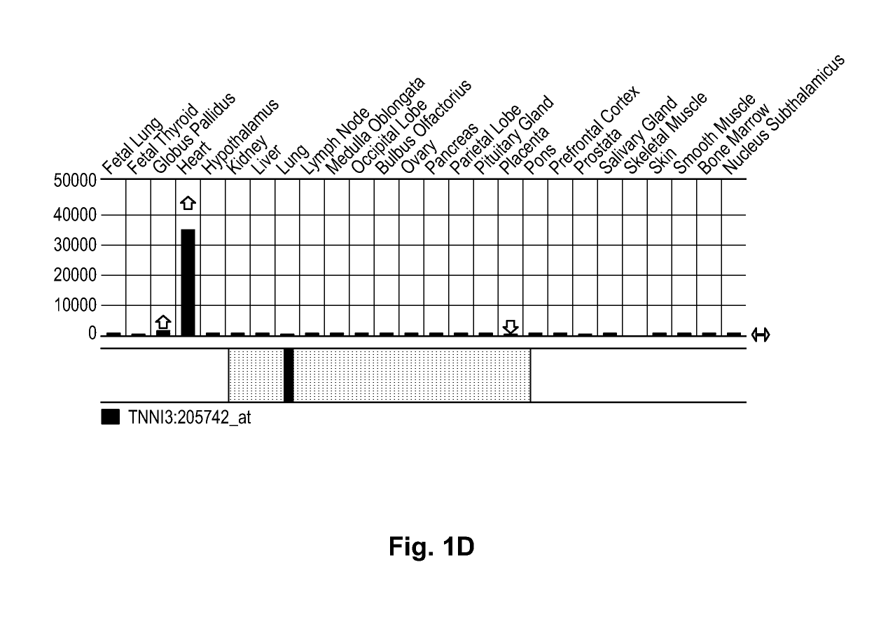

The obtained protein identifications were searched for heart-specific proteins. They are either known from physiological text book literature, e.g. by considering the special muscle functionality of the heart, or were filtered by a comparison with expression databases, e.g. the Gene Expression Atlas, operated by the European Institute for Bioinformatics and accessible via UniProt. The diagrams shown in FIG. 1 depict the mRNA expression patterns of the four proteins which are primarily responsible for the heart-specific signature. It is to be noted that the specific signature is composed by proteins which almost exclusively prevail in cardiac tissue such as myosin binding protein C (MYPC3, FIG. 1A) and troponin I (TNNI3, FIG. 1 D), and of such proteins which have secondary specificities such as troponin C (TNNC1, FIG. 1 B) and myosin regulatory light chain 2 (MLRV, FIG. 1C).

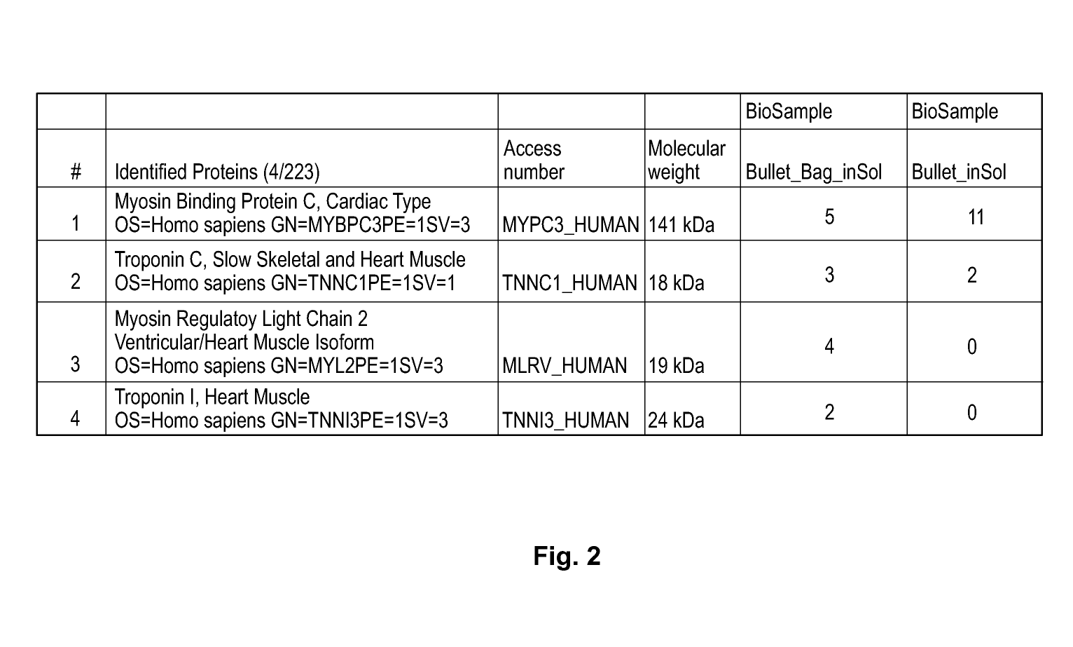

The result of such preliminary experiment is shown in FIG. 2. There the analyzed heart-specific protein signature is depicted consisting of the four proteins of myosin binding protein C (MYPC3), troponin C (TNNC1), myosin regulatory light chain 2 (MLRV), and troponin I (TNNI3). The detection of these four heart-specific proteins in the biological sample allows a very specific allocation of the bullet to the organ heart.

In the course of this preliminary experiment it could be demonstrated that on the basis of the determination of a heart-specific protein signature of the biological material on the surface of a bullet, the organ-specific penetration, in particular of the heart, can be allocated.

### Example 3

### Main Experiment

In a blinded experimental approach different bullets were analyzed which before have penetrated human organs.

Sample Isolation/Preparation

Eight different bullets which before have penetrated eight different human organs were given into test tubes and stored at −20° C. until the further processing. For the analysis the metal balls were removed and subjected to a proteolysis in suitable plastic vials by the aid of the protease trypsin. For this purpose in a solution a reduction with dithiothreitol and an alkylation with iodoacetamide were realized and in the following the protease was added and an incubation over night at 37° C. was performed. The reactions were stopped by an acidification with trifluoroacetic acid (final concentration: 5%) and the resulting peptide solutions were centrifuged. After the concentration via stage tips (Proxeon) a small part of the supernatant was separated on an UltiMate 3000 Nano HPLC installation (Dionex) via a Acclaim PepMap RSLC 75μ×25 cm C18 2 μm 100 A.

Establishing a Total Protein Profile by Mass Spectrometry

The concentrated and chromatographically separated supernatants of the peptide solutions were measured online in a LTQ Orbitrap mass spectrometer (Thermo Fischer Scientific). In each case the 10 most intensive masses of a filling cycle were automatically selected for a fragmentation. The fragmentation was done by means of the CID method (collision induced decay). The masses appearing in the fragment spectra were compared with the theoretic protein and peptide mass parameters from the SwissProt databse by using the Mascot search algorithm (Matrix Science) and the results were visualized by the aid of the Scaffold software (Proteome Software Inc.). The result is a total protein profile in the form of so-called hit or result lists where the protein identifications were weighted with respect to their reliability or non-accidental appearance.

Analysis of the Total Protein Profile for Organ-Specific Protein Signatures

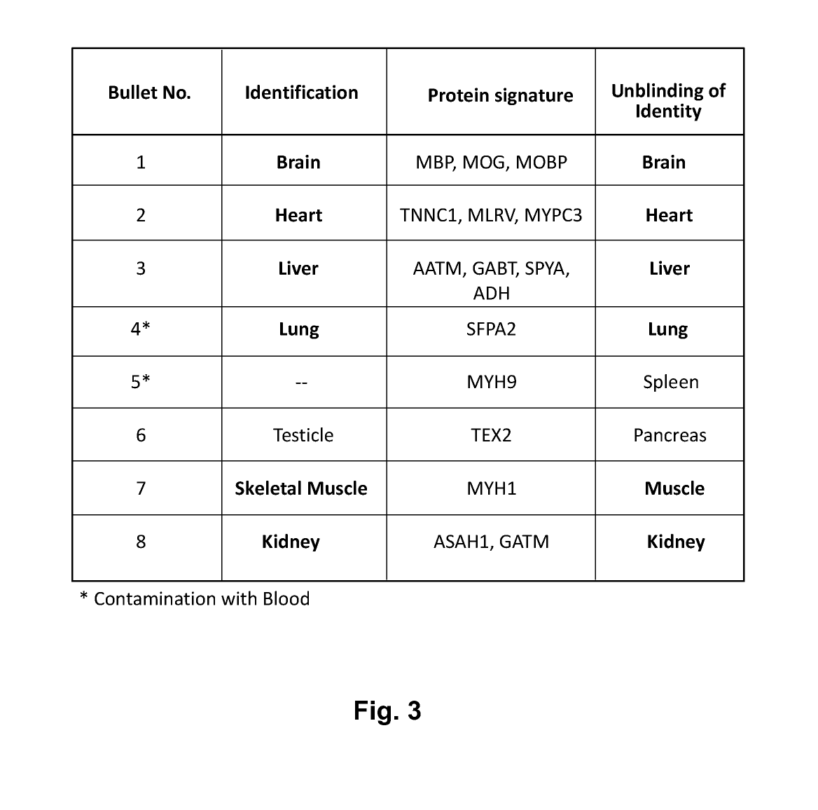

The result of the main experiment is shown in FIG. 3. Such identifications are printed in bold which could be correctly allocated (true identifies shown in the right column). Samples labeled with * (star) were not correctly provided, in particular because of a strong contamination with blood.

The outcome of this is that six out of eight bullets could be correctly allocated to the organs which were in fact penetrated by them. It was succeeded in a correct allocation to the organs of brain (bullet No. 1), heart (No. 2), liver (No. 3), lung (No. 4), skeletal muscle (No. 7), and kidney (No. 8). The bullet No. 5 was heavily contaminated with blood and could not be correctly allocated. Bullet No. 6 could also not be correctly allocated. This results in a hit rate of 75% including the heavily contaminated bullets, and of more than 83% under consideration of exclusively correctly provided bullets.

### CONCLUSION

The inventors succeeded in providing a forensic method for the identification of an organ penetrated by a corpus delicti, which is characterized by a high specificity, a simple handling and a very high hit rate.

What is claimed is:

1. Forensic method for identifying an organ penetrated by a corpus deliciti, comprising the following steps:
1) providing of biological material which was in contact with the corpus delicti,
2) establishing of a total protein profile of the biological material,
3) analyzing of the total protein profile for an organ-specific protein signature,
4) identifying of the organ penetrated by the corpus delicti in case of the presence of an organ-specific protein profile.

2. Forensic method of claim 1, wherein the organ-specific protein signature is selected from the group consisting of: brain-specific, heart-specific, liver-specific, lung-specific, kidney-specific, and skeletal muscle-specific protein signature.

3. Forensic method of claim 1, wherein the organ-specific protein signature comprises at least 2 to at least 10 or more organ-specific proteins.

4. Forensic method of claim 3, wherein the
brain-specific protein signature comprises brain-specific proteins which are selected from the group consisting of: myelin basic protein (MBP), myelin oligodendrocyte glycoprotein (MOG), myelin-associated oligodendrocytic basic glycoprotein (MOBP), and the AT1 A3-Na/K-ATPase-alpha-3-subunit, and/or
heart-specific protein signature comprises heart-specific proteins which are selected from the group consisting of: myosin binding protein C (MYBPC3), troponin C (TNNC1), ventricular/myocardial muscle isoform of the myosin regulatory light chain 2 (MYL2), troponin I (TNNI3), AT1A1-Na/K-ATPase-alpha-1-subunit (AT1A1), creatin kinase S type (KCRS), citrate synthase (CISY), and isocitrate dehydrogenase (IDHP), and/or the
liver-specific protein signature comprises liver-specific proteins which are selected from the group consisting of: aspartate aminotransferase (AATM), 4-aminobutyrate aminotransferase (GABT), serine-pyruvate aminotransferase (SPYA), alcohol dehydrogenase (ADH), carbamoyl-phosphate synthase (CPSM), glycogen phosphorylase (PYGL), copper transport protein (ATOX1), phospholysine phosphohistidine inorganic pyrophosphate phosphatase (LHPP), proteasome subunit alpha type-2 (PSMA2), proteasome subunit alpha type-6 (PSMA6), glycine N-acyltransferase (GLYAT), glycine amidinotransferase (GATM), arginase 1 (ARGI1), prostaglandine F synthase (PGFS), prostaglandine reductase (PTGR1), and argininosuccinate lyase (ARLY), and/or the
lung-specific protein signature comprises lung-specific proteins which are selected from the group consisting of: pulmonary surfactant associated protein A1 (SFPA1), pulmonary surfactant associated protein A1 (SFPA2), pulmonary surfactant associated protein B (PSPB), cathelicidine antimicrobial peptide (CAMP), annexin A1/A3/A2 (ANXA), transgelin 2 (TAGLN), histones H1, H2b, H4 (H2, H4), 14-3-3 beta/alpha/delta (1433B), and/or the
skeletal muscle-specific protein signature comprises skeletal muscle specific proteins which are selected from the group consisting of: actin/alpha skeletal muscle (ACTA), troponin C/skeletal muscle (TNNC), troponin T/fast skeletal muscle (TNNT), myosin light-chain ⅓ skeletal muscle isoform (MLC), myosin regulatory light-chain 2 skeletal muscle isoform (MLRS), myosin 1 (MYH1), myosin 2 (MYH2), aldolase C fructose-bisphosphate (ALDOC), aldolase A fructose-bisphosphate (ALDO), myoglobin (MYG), and/or the
kidney-specific protein signature comprises kidney-specific proteins selected from the group consisting of: acid ceramidase (ASAH1), glycine amidinotransferase (GATM), chloride intracellular channel protein (CLIC4), transketolase (TKT), voltage-dependent anion-selective channel (VDAC), aquaporine 1 (AQP1).

5. Forensic method of claim 1, wherein steps 2 and 3 are performed by means of a mass spectrometric analysis.

6. Forensic method of claim 5, wherein the total protein profile of the biological material is established in step 2 as follows:
analyzing by mass spectrometry of the total protein of the biological material to obtain mass spectra, and
comparing of the obtained mass spectra with a protein database to obtain the total protein profile in form of a pattern which identifies individual proteins.

7. Forensic method of claim 6, wherein the pattern which identifies individual proteins is analyzed for the presence of the organ-specific protein signature.
